(19) United States
(12) Patent Application Publication
(10) Pub. No.: US 2024/0068969 A1
(43) Pub. Date: Feb. 29, 2024

(54) SENSOR, CHANNEL, AND FUEL CELL SYSTEM

(71) Applicant: Vitesco Technologies GmbH, Regensburg (DE)

(72) Inventors: Stephan HEINRICH, Pfeffenhausen (DE); Wolfgang LAUERER, Laaber (DE); Rainer FRAUENHOLZ, Pettendorf (DE); Stephen SETESCAK, Pentling (DE); Stefan PESAHL, Saal an der Donau (DE)

(21) Appl. No.: 18/237,983

(22) Filed: Aug. 25, 2023

(30) Foreign Application Priority Data
Aug. 26, 2022 (DE) 102022208864.6

(51) Int. Cl. G01N 25/18; G01N 33/00; H01M 8/0444

(52) U.S. Cl. CPC G01N 25/18; G01N 33/005; H01M 8/04462; H01M 2250/20

(57) ABSTRACT

A sensor for determining a hydrogen concentration in a channel of a fuel cell system, having a sensor housing, a measuring chamber, wherein the measuring chamber is arranged in the sensor housing, and a measuring chamber channel, wherein the measuring chamber channel fluidically connects the measuring chamber to the environment of the sensor, wherein the sensor can be mounted in such a way that the measuring chamber diaphragm is located in the channel. A channel having a sensor of this type and a fuel cell system having a channel of said type.

### BACKGROUND OF INVENTION

### 1. Field of the Invention

The disclosure relates to a sensor, a channel having such a sensor, and a fuel cell system having such a channel.

### 2. Description of Related Art

Sensors for determining the hydrogen concentration in a channel of a fuel cell system are usually located outside the channel so that they can be decoupled from the difficult measurement conditions within the channel. For this purpose, it is usually necessary to provide a heating process before each commissioning of the sensor in order to avoid moisture that could affect the measuring process

U.S. Pat. No. 6,668,616 B1 discloses a carbon monoxide sensor.

A disadvantage of the devices in the prior art is in particular the effort that is required before each commissioning in order to avoid any effect of moisture on the measuring process.

### SUMMARY OF THE INVENTION

An object of one aspect of the present invention is to provide an alternative sensor, which is characterized in particular in that no heating process is required before a measuring process with the sensor is commissioned. A further object consists in providing a channel having a sensor of this type. Furthermore, a further object consists in providing a fuel cell system having a channel of this type.

One aspect of the invention relates to a sensor for determining a hydrogen concentration in a channel of a fuel cell system, wherein the sensor comprises a sensor housing, a measuring chamber, wherein the measuring chamber is arranged in the sensor housing, and a measuring chamber channel, wherein the measuring chamber channel fluidically connects the measuring chamber to the environment of the sensor, wherein the sensor can be mounted in such a way that the measuring chamber is located in the channel.

The fact that the sensor can be mounted in such a way that the measuring chamber is located in the channel removes the need to perform a separate heating process before commissioning the sensor, because the flow in the channel can be used to heat up the sensor. This avoids any negative effect on the measurement process by the moisture, even without a separate heating process before commissioning the sensor.

It is particularly advantageous if the sensor is a thermal sensor. In other words, the sensor comprises a heating element and a temperature sensor, which are arranged in particular within the measuring chamber. Depending on the hydrogen concentration in the measuring chamber, during a heating process by the heating element, a temperature curve can be determined by the temperature sensor, from which the hydrogen concentration in the measuring chamber can be determined. This measurement process is based mainly on the high thermal capacity of hydrogen compared to the other components of the gas, the hydrogen concentration of which must be determined.

It is also advantageous if the channel is an exhaust gas channel of a fuel cell system. It is furthermore advantageous if the fuel cell system is a fuel cell system for a motor vehicle.

It is particularly advantageous if the measuring chamber channel fluidically connects the measuring chamber to the environment of the sensor housing.

In addition, it is preferred if the sensor can be mounted in such a way that the measuring chamber channel and/or the sensor housing are arranged in the channel.

It is particularly advantageous if the sensor can be mounted in such a way that the measuring chamber channel fluidically connects the measuring chamber to the channel.

It is particularly preferred if the measuring chamber is fluidically connectable to the channel by the measuring chamber channel.

A preferred exemplary embodiment is characterized in that the measuring chamber channel is closed by a diaphragm and that the diaphragm is gas-permeable and fluid-impermeable. This reliably prevents the entry of fluid from the channel into the measuring chamber, thereby further reducing the moisture inside the measuring chamber.

It is also preferable if the diaphragm is connected or fixed to the sensor housing in a materially bonded manner, for example by adhesive bonding or welding. In particular, the connection or fixing is designed in such a way that a bypass current past the diaphragm into the measuring chamber is prevented. This prevents fluid from entering the measuring chamber.

In addition, it is advantageous if the sensor can be mounted in such a way that the diaphragm is arranged in the channel.

Another aspect is characterized in that the measuring chamber channel feeds into a measuring chamber opening and that the measuring chamber opening is arranged on the surface of the sensor housing.

A further aspect is characterized in that the measuring chamber opening is closed off by the diaphragm. This prevents an ingress of fluid into the measuring chamber channel. At the same time, the diaphragm is arranged on the surface of the sensor housing and is thus arranged in such a way, when the sensor is arranged in the channel, that the flow inside the channel cleans the diaphragm and blows away any liquid droplets. In addition, the installation or fixing of the diaphragm is particularly simple with this arrangement of the diaphragm.

Another aspect is characterized in that the sensor comprises a flow-influencing geometry that influences the flow in the channel.

It is also preferable if the flow-influencing geometry is arranged in the channel against the flow direction of the flow when viewed from the measuring chamber opening, when the sensor is mounted.

In addition, it is advantageous if the flow-influencing geometry is integral, in particular formed in one piece with the sensor housing. This is a very cost-effective solution.

Furthermore, it is advantageous if the flow-influencing geometry is directly adjacent to the measuring chamber opening.

It is also convenient if the flow-influencing geometry is designed such that when the sensor is mounted, the flow in the channel is diverted to the measuring chamber opening and/or to the diaphragm. This will keep the measuring chamber opening and/or diaphragm clean due to the flow.

In addition, it is advantageous if the flow-influencing geometry is designed in such a way that when the sensor is mounted, the flow-influencing geometry generates a turbulent flow from the flow in the channel. This will keep the measuring chamber opening and/or the diaphragm clean due to the flow.

One aspect is characterized by the fact that the measuring chamber channel is linear, angled or formed in the shape of a labyrinth. While a linear design is particularly inexpensive and simple, particularly if manufactured by plastic injection molding, an angled design or a labyrinth-shaped design is particularly helpful in preventing the entry of fluid into the measuring chamber, in particular in the case of a damaged or absent diaphragm.

An object with respect to the channel is provided by a channel for a fuel cell system having a sensor according to one aspect of the invention, wherein the sensor is arranged in the channel Preferably, the sensor is located partially or completely in the channel. It is furthermore preferable if the measuring chamber opening is located in the channel. In addition, it is preferable if the measuring chamber channel is completely or partially located in the channel. It is particularly advantageous if the sensor housing is partially or completely located in the channel. It is preferred furthermore if the channel is an exhaust gas channel. In addition, the channel is preferably circular or has a circular cross-section.

In addition, it is advantageous if the channel is made of a metal, in particular stainless steel, or a plastic.

It is particularly preferred if the measuring chamber channel fluidically connects the measuring chamber to the channel.

One aspect of the invention is characterized in that the sensor housing, the measuring chamber, the measuring chamber channel, the diaphragm, and/or the measuring chamber opening is intersected by an area centroid vector, wherein the area centroid vector passes through the area centroid of a channel cross-section of the channel, wherein the channel cross-section is located in a plane perpendicular to the longitudinal direction of the channel, and intersects the sensor, and that the area centroid vector intersects the plane at right angles. Furthermore, it is particularly preferred if the area centroid vector intersects at least one, two, three, four or five of the following components of the sensor: the sensor housing, the measuring chamber, the measuring chamber channel, the diaphragm, and the measuring chamber opening.

It is particularly preferred if the area centroid vector is spaced apart from at least one, two, three, four or five of the following components of the sensor: the sensor housing, the measuring chamber, the measuring chamber channel, the diaphragm, and the measuring chamber opening. In other words, the components of the sensor are either intersected by or spaced apart from the vector. A spaced apart arrangement means that the component is not intersected by the vector. In particular, the longitudinal direction refers to the flow direction of the gas within the channel, in which the gas can flow, or the extension direction of the channel.

It is also advantageous if the channel is an exhaust gas channel of a fuel cell system. It is furthermore advantageous if the fuel cell system is a fuel cell system for a motor vehicle.

One aspect of the invention is characterized in that the sensor is arranged in the channel in such a way that the area centroid vector intersects a further plane, in which the diaphragm or the measuring chamber opening is arranged, at an angle of 70° to 110°.

One aspect of the invention is characterized in that the sensor is arranged in such a way that the diaphragm is at least partially opposed to the flow direction. In other words, the sensor is arranged in such a way that the diaphragm faces the flow in the channel. This arrangement makes it possible for the flow to clean the diaphragm or remove droplets from the diaphragm.

One aspect of the invention is characterized in that the sensor is fixed to a channel wall of the channel. It is particularly preferred if the sensor is fixed by a thread pair comprising an external thread on the sensor housing and an internal thread in the channel wall. In this way, mounting the sensor is particularly simple and time-saving.

Furthermore, it is particularly preferred if the sensor housing has thermal insulation at the mounting point of the sensor with the channel wall. This prevents the sensor from cooling due to the temperature of the channel wall. This helps prevent any droplets from accumulating on the sensor or sensor housing.

One aspect of the invention is characterized in that the sensor extends from outside the channel into the channel through an opening in the channel wall. In this case, it is possible that the sensor is flanged to the channel wall.

An object with regard to the fuel cell system is achieved by way of a fuel cell system having a channel according to the invention. This creates a fuel cell system which can dispense with a separate operation before commissioning the sensor. It is furthermore advantageous if the fuel cell system is a fuel cell system for a motor vehicle.

In addition, it is particularly preferable to provide a motor vehicle having such a fuel cell system.

Advantageous developments of the present invention are described in the subclaims and in the description of the figures that follows.

### BRIEF DESCRIPTION OF THE DRAWINGS

The invention is explained in detail below on the basis of exemplary embodiments with reference to the drawings, in which:

FIG. 1 is a motor vehicle having a fuel cell system;

FIG. 2 is a channel with a sensor; and

FIG. 3 s a channel with a sensor.

### DETAILED DESCRIPTION OF THE PRESENTLY PREFERRED EMBODIMENTS

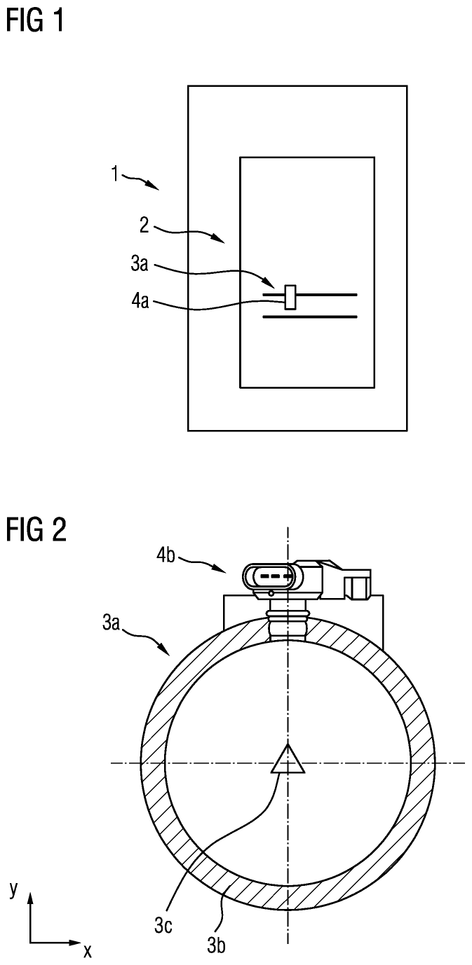

FIG. 1 shows a motor vehicle 1 that comprises a fuel cell system 2 according to one aspect of the invention. The motor vehicle 1 has an electric drive. The fuel cell system 2 comprises a channel 3a with a sensor 4a according to one aspect of the invention, designed as a thermal sensor 4a, to determine the hydrogen concentration in the channel 3a.

FIG. 2 shows a sectional view which is perpendicular to the flow path within the channel 3a and perpendicular to the extension direction of the channel. In other words, the channel cross-section shown lies in a plane perpendicular to the longitudinal direction of the channel 3a. This is the X-Y plane. The channel 3a has a circular cross-section, the area centroid 3c of which is located in the center of the circular cross-section of the channel 3a. In addition, the channel 3a has a channel wall 3b made of stainless steel, with a constant wall thickness. Furthermore, a sensor 4b known from the prior art for determining the hydrogen concentration is shown, which is located outside the channel 3a and is fixed to the channel wall 3b. The sensor 4b is fluidically coupled to the channel 3a through an opening in the channel wall 3b. Since sensor 4b is located outside the channel 3a, the sensor 4b is largely decoupled from the temperature of the flow in channel 3a.

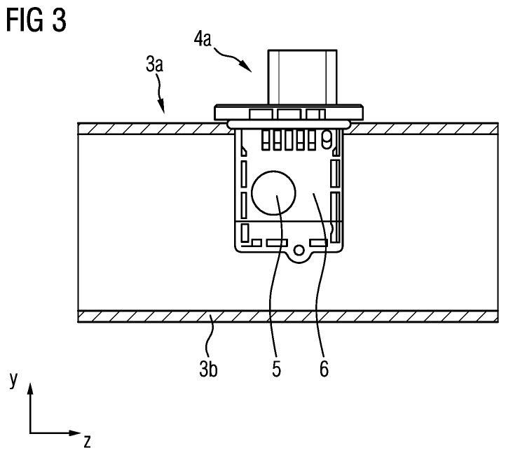

FIG. 3 shows the same circular channel 3a from FIG. 2, but with a sensor 4a according to one aspect of the invention which is attached to the channel wall 3b and extends from outside the channel 3a to the interior of the channel 3a through an opening in the channel wall 3b. The view shown is a longitudinal section through the channel 3a in the Z-Y plane. The channel 3a extends in the Z direction, whereby the flow within the channel 3a also runs in the Z direction. The sensor 4a shown here is the sensor 4a, which is shown in FIG. 1. The sensor 4a is attached to the channel wall 3b by a thread, not shown in more detail here, and also has an elastomer seal to prevent the escape of gas within the channel. The sensor 4a comprises a sensor housing 6, in which a measuring chamber is accommodated. A heating element and a temperature sensor are accommodated in the measuring chamber to measure the concentration of hydrogen in the measuring chamber. The measuring chamber is fluidically coupled to the channel by a measuring chamber channel which feeds into a measuring chamber opening. The measuring chamber opening is arranged on the surface of the sensor housing 6 and is covered by a diaphragm 5. The diaphragm 5 is gas-permeable and fluid-impermeable. The sensor housing 6 is made of a material that has a high thermal conductivity.

The exemplary embodiments in FIGS. 1 and 3 are in particular not of a limiting nature and serve for illustrating the concept of the invention.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A sensor configured to determine a hydrogen concentration in a channel of a fuel cell system, comprising:
a sensor housing;
a measuring chamber arranged in the sensor housing; and
a measuring chamber channel that fluidically connects the measuring chamber to an environment of the sensor,
wherein the sensor is configured to be mounted such that the measuring chamber is located in the channel.

2. The sensor as claimed in claim 1, further comprising:
a diaphragm that is gas-permeable and fluid-impermeable and configured to close the measuring chamber channel.

3. The sensor as claimed in claim 1, wherein the measuring chamber channel feeds into a measuring chamber opening and that the measuring chamber opening is arranged on a surface of the sensor housing.

4. The sensor as claimed in claim 3, wherein the measuring chamber opening is closed by a diaphragm that is gas-permeable and fluid-impermeable.

5. The sensor as claimed in claim 1, wherein the sensor comprises a flow-influencing geometry that influences a flow in the channel.

6. The sensor as claimed in claim 1, wherein the measuring chamber channel is linear, angled, or a labyrinth.

7. A channel for a fuel cell system, comprising:
a sensor arranged in the channel and configured to determine a hydrogen concentration in the channel of the fuel cell system, comprising:
a sensor housing;
a measuring chamber arranged in the sensor housing; and
a measuring chamber channel that fluidically connects the measuring chamber to an environment of the sensor,
wherein the sensor is configured to be mounted such that the measuring chamber is located in the channel.

8. The channel as claimed in claim 7,
wherein the sensor housing, the measuring chamber, a diaphragm that is gas-permeable and fluid-impermeable and configured to close the measuring chamber channel, the measuring chamber channel, and/or a measuring chamber opening is intersected by an area centroid vector, and
wherein the area centroid vector passes through an area centroid of a channel cross-section of the channel,
wherein the channel cross-section is located in a plane perpendicular to a longitudinal direction of the channel, and intersects the sensor, and that the area centroid vector intersects the plane at right angles.

9. The channel as claimed in claim 8, wherein the sensor is arranged in the channel such that the area centroid vector intersects a further plane, in which the diaphragm or the measuring chamber opening is arranged, at an angle of 70° to 110°.

10. The channel as claimed in claim 7, wherein the sensor is arranged in such a way that a diaphragm that is gas-permeable and fluid-impermeable and configured to close the measuring chamber channel is at least partially opposed to a flow direction.

11. The channel as claimed in claim 7, wherein the sensor is attached to a channel wall of the channel.

12. The channel as claimed in claim 7, wherein the sensor extends from outside the channel into the channel through an opening in a channel wall.

13. A fuel cell system comprising:
a channel, comprising:
a sensor arranged in the channel and configured to determine a hydrogen concentration in the channel of the fuel cell system, comprising:
a sensor housing;
a measuring chamber arranged in the sensor housing; and
a measuring chamber channel that fluidically connects the measuring chamber to an environment of the sensor,
wherein the sensor is configured to be mounted such that the measuring chamber is located in the channel.
